(19) United States
(12) Patent Application Publication
(10) Pub. No.: US 2023/0404233 A1
(43) Pub. Date: Dec. 21, 2023

(54) DEVICES AND TECHNIQUES FOR STYLING HAIR

(71) Applicant: MIRAKEL TECHNOLOGIES, INC., CAMBRIDGE, MA (US)

(72) Inventors: Pradeep Paul PANENGAD, Bukit Panjang (SG); Michael STERN, Natick, MA (US)

(21) Appl. No.: 18/030,330

(22) PCT Filed: Oct. 5, 2021
(86) PCT No.: PCT/US2021/053464

§ 371 Date: Apr. 5, 2023

Related U.S. Application Data
(60) Provisional application No. 63/087,914, filed on Oct. 6, 2020.

(51) Int. Cl. A45D 19/00; A45D 19/02

(52) U.S. Cl. CPC A45D 19/012; A45D 7/00; A45D 19/02; A45D 19/0066

(57) ABSTRACT

In various embodiments, hairstyling devices utilize liquid agents for styling, bleaching, coloring, and/or other hair treatments. Leakage of the liquid agents is prevented by use of a vacuum source that removes excess liquid from the device, sufficient liquid for treatment remaining within the device in one or more absorbent pads.

### RELATED APPLICATIONS

This application claims the benefit of and priority to U.S. Provisional Patent Application No. 63/087,914, filed Oct. 6, 2020, the entire disclosure of which is hereby incorporated herein by reference.

### TECHNICAL FIELD

In various embodiments, the present invention relates to the electrochemical styling of hair utilizing a handheld device.

### BACKGROUND

Hair is made of a protein keratin, which is rich in sulfur containing amino acids. The keratin protein matrix in the hair is held together by millions of disulfide bonds that provide the strength and stability of the hair. The disulfide bonds located on the surface of the hair have the maximum leverage in determining the shape of the hair. The distribution of the disulfide bonds with respect to the axis of the hair can dictate the “straightness” or “curliness” of the hair. The disulfide bonds act as a scaffold and can be repositioned to alter the condition and/or orientation of the hair. Conventional methods for styling hair such as, for example, “hair rebonding” and “perming techniques” use heat and/or chemicals to break the disulfide bonds and shape the hair into the desired form, for example, straight or curled, and subsequently rebond the disulfide bonds, such that the hair retains the reshaped form for some period of time.

Conventional methods for styling hair have several disadvantages. For example, the heat and/or chemicals used in conventional hair styling devices and methods can cause significant damage to the hair-root and scalp, can take a long time for the styling operation, and often require the assistance of an expensive professional hairdresser. Many of these disadvantages have been addressed with a recent new class of devices that may be used to style hair electrochemically with non-toxic styling agents. Examples of such devices are disclosed in U.S. Pat. No. 9,687,056, filed on Aug. 22, 2014 (the '056 patent), and U.S. Patent Application Publication No. 2017/0071312, filed on Aug. 4, 2016 (the '1312 application), the entire disclosures of which are hereby incorporated by reference herein. These devices produce a highly localized alkaline or acidic “electrolysis zone” in an electrolyte located in a handpiece. Hair is exposed to the alkaline and/or acidic zones produced in the electrolyte to alter the sulfhydryl, ionic, and/or hydrogen bonds included in the hair, and thus the hair may be styled using electrolysis via the handpiece.

However, while electrochemical hair-styling devices address many issues related to conventional hair styling, these devices may still exhibit shortcomings. Specifically, the liquid styling agent may leak from the handpiece, resulting in unsightly stains on the clothing of the subject and/or the floor below the work area. Such leakage may hinder the widespread adoption of electrochemical handheld hair-styling devices, and thus there is a need for improved devices that minimize or eliminate leakage of the liquid styling agent.

### SUMMARY

In accordance with various embodiments of the present invention, handheld hair-styling devices utilize a liquid agent, and optionally with electrolysis, to style hair. Devices in accordance with embodiments of the invention include a handpiece with two electrodes (herein also referred to as the “anode” and “cathode”). An electrical potential difference between the two electrodes produces an electrolysis zone in the liquid agent that is utilized to style hair. The handpiece is fluidly connected to one or more holding tanks or reservoirs for the liquid styling agent (e.g., an electrolyte) or liquid waste, which may be pumped into and out of the handpiece by one or more pumps. Herein, “anode” and “cathode” (and “anode-side” and “cathode-side”) refer to opposing electrodes that are typically biased at different electrical potentials. In some embodiments, the potentials of the electrodes may be switched, to allow for the production of either alkaline or acidic solutions by electrolysis (e.g., using the same electrolyte).

Electrolysis is the process of using a direct electrical current to facilitate a chemical reaction, which involves driving ionic current through a liquid, for example, an aqueous electrolyte. This may be achieved by contacting two electrodes having a liquid disposed therebetween and imposing a difference in electrical potential on the two electrodes. For example, one of the electrodes may be negatively charged while the other is positively charged. In this manner, an “electrolysis zone” may be formed between the two electrodes. At a negatively charged electrode (e.g., a “cathode”), water molecules included in the aqueous electrolyte decompose to produce H⁺ and OH⁻ (i.e., “in situ”). The H⁺ may be consumed by reduction to hydrogen gas such that a region surrounding the cathode is highly reducing and possesses an alkaline pH (e.g., a pH greater than about 12 or of about 12-14). In some embodiments, an electrode may be positively charged, in which case water molecules of the liquid decompose to produce H⁺ and OH⁻. The OH⁻ may be consumed by oxidation to oxygen gas, and the free H⁺ ions may combine with a water molecule to produce H₃O⁺ (hydronium) ions, such that a region proximate to the anode has a highly acidic and oxidizing pH (e.g., a pH less than 4, of about 0-4, or of about 1-4). In some embodiments, a buffer may be added to control the pH. In some embodiments, an additional component may be added to the electrolyte to react at the cathode or anode instead of or in addition to the electrolysis of water.

In various embodiments, the hairstyling device features two opposing electrodes (i.e., an anode and a cathode), which may be brought in close proximity to hair (e.g., human hair) to produce the electrolysis zone in the vicinity of the hair. This may allow the hair to undergo physical changes. For example, when exposed to an alkaline electrolysis zone, the hair swells. Without being bound by any particular theory, the swelling of the hair may be attributed to the breakdown of various bonds, e.g., the disulfide bonds that fasten the hair proteins together, ionic bonds, and/or hydrogen bonds. For example, an untreated portion of a hair may include a plurality of disulfide bonds, ionic bonds, and hydrogen bonds randomly distributed in the hair. After exposure to an alkaline electrolysis zone, for example, having a pH of at least 10 (e.g., at least about 11, at least about 12, or at least about 13), produced by a cathode (i.e., a negatively charged electrode), the distribution of bonds within the hair may change, for example because the reducing environment of the alkaline electrolysis zone may open the cuticle of the hair and break a substantial portion of the disulfide bonds and, when coupled with mechanical stretching (e.g., straightening) of the portion of the hair by the electrodes, may achieve straightening of the hair. Many of the disulfide bonds may undergo lanthionization as well. However, a small portion of the disulfide bonds may persist. If subsequently exposed to a neutral pH environment, the disulfide bonds, the ionic bonds, and/or the hydrogen bonds may reform, and the cuticle may close. In this manner, the portion (or “bundle”) of the hair may be styled. For example, exposing hair to an alkaline environment may cause it to transition from a wavy or curly shape into a straight or substantially straightened shape. Furthermore, the dense keratin network of hair may readily open up when exposed to an alkaline pH. This may allow permeation of the hair with various molecules, for example, colors, dyes, anti-frizz agents, oils, straighteners, moisturizers, shape memory materials, nanoparticles, monomers that favor polymerization (e.g., photo- or light-induced polymerization), molecular assemblies (e.g., micelles for coloring, anti-frizzing, enhancing shine and other treatments of hair) or any other molecules used for styling and/or treating hair.

On the other hand, when a portion of hair is exposed to an acidic electrolysis zone, the hair contracts, possibly because of reformation of bonds, new bonds, and/or crosslinking which may fasten the hair proteins together. Thus, whether an electrolysis zone (or region thereof) produced between the opposing electrodes is alkaline or acidic may determine a corresponding reconfiguration of the location and/or structure of all the bonds stabilizing the hair structure (i.e., the location of the disulfide bonds included in the hair) and, thereby, restyle the hair. For example, exposing hair to an acidic environment may cause it to transition from frizzy to smooth or non-frizzy.

Thus, various embodiments of the present invention are utilized for electrolytic or electrolysis-based bond multiplication of hair, which recovers the strength of the hair after any of various treatments that can weaken the hair. Exposing the hair to an acidic pH may multiply or increase the different types of bonds within the hair to strengthen it. For example, various types of bonds naturally present in the hair may be reformed, various hair compounds may be cross-linked, and/or polymeric backbones in the hair may be built up.

Devices in accordance with various embodiments of the invention may employ any of a variety of different styling agents (or “electrolytes”) to facilitate formation of the electrolysis zone that may be utilized to style hair therein. Exemplary styling agents include, but are not limited to, solutions of water and one or more salts. The salt concentration (i.e., molarity, “M”) is preferably relatively high, such that a desired level of anion is present within the electrolyte during use. The higher salt concentration may increase the conductivity of the electrolyte and, in turn, can increase the OH⁻ ion concentration. Higher salt concentrations and conductivity may result in higher currents or lower voltages during use. For example, the salt concentration may be about 2 M, or greater than about 2 M, or about 3 M, or from about 1 M to about 5 M, or at least about 2.5 M, or from about 2.5 M to about 3.5 M, or from about 2 M to about 3 M, or from about 1.8 M to about 2.5 M. The maximum molar concentration may depend on the solubility of the particular salt. The salt may be any suitable salt that is safe for human exposure, for example, including lithium, sodium, potassium, calcium, or magnesium salts. Exemplary salts can include anions such as carbonate, bicarbonate, maleate, halides such as chloride or bromide, phosphate, sulfate, and nitrate, among others. Exemplary salts include lithium sulfate, sodium bicarbonate, sodium chloride (NaCl), sodium carbonate, sodium sulfate, sodium iodide (NaI), sodium maleate, magnesium sulfate, magnesium chloride, calcium chloride, potassium chloride, potassium iodide, potassium sulfate, potassium bicarbonate, potassium carbonate, sodium acetate, sodium citrate, sodium phosphate, chromium chloride, aluminum chloride, Epsom salt, any other suitable salt or combination thereof. In some embodiments, the salt is compatible with production of both alkaline and acidic electrolyzed solutions. In various embodiments, styling agents may include, consist essentially of, or consist of one or more of the electrolytes disclosed in the '056 patent and/or the '1312 application.

In addition to salts, styling agents in accordance with various embodiments of the present invention may include agents that promote the lanthionization of the sulfide bonds, for example maleic acid and/or maleic anhydride. Styling agents may also include any salts (e.g., salts including and/or fabricated using one or more alkali metals and/or alkali earth metals) of organic acids, e.g., sodium maleate and/or sodium citrate. Furthermore, rheology modifiers may be added to increase the viscosity of the styling agent. In some embodiments, more than one styling agent may be used. For example, a system may use one styling agent at the cathode (a “catholyte”) and a separate agent at the anode (an “anolyte”). In various embodiments, when alkaline styling agents are utilized, the pH of the styling agent is at least 10, at least 11, at least 12, or at least 13.

In additional embodiments, the styling system may supply a liquid styling agent that is already rich in hydroxide ions in addition to or instead of performing electrolysis in the handpiece. For example, the styling agent may include, consist essentially of, or consist of an alkaline liquid such as sodium hydroxide. Application of the styling agent to a bundle of hair with the handpiece enables styling of the hair. For example, an alkaline liquid can be dispensed during one or more strokes of a bundle of hair using the device to effect hair straightening, followed by dispensing and application of an acidic solution to the same bundle of hair by one or more strokes with the device to neutralize the alkaline pH. In such embodiments, the handpiece may lack one or both electrodes and may simply include one or more pads for application of the styling agent to hair within the handpiece. In various embodiments, the flow of the fluid to and through the handpiece may be controlled via one or more sensors in or proximate the handpiece that detect ionic resistance or conductivity to ensure sufficient flow of the styling agent for the desired hair styling.

In yet additional embodiments, the styling agent to be utilized for hair styling undergoes electrolysis within an electrolysis chamber or reservoir that is not within, but that is fluidly connected to, the handpiece. In such embodiments, the electrolyzed solution may be pumped to the handpiece for application to the hair after undergoing electrolysis. In such embodiments, the handpiece may lack one or both electrodes and may simply include one or more pads for application of the styling agent to hair within the handpiece. The electrolysis apparatus may have controls to switch between producing alkaline and acidic solutions, allowing for convenient shifting between hair straightening and neutralization modes.

In an aspect, embodiments of the invention feature a hairstyling device that includes, consists essentially of, or consists of an anode-side housing having an outer perimeter, an electrically conductive anode disposed within the anode-side housing, a cathode-side housing having an outer perimeter, an electrically conductive cathode disposed within the cathode-side housing, a membrane disposed between the anode and the cathode, an absorbent pad disposed adjacent to the membrane, one or more fluid inlets for supplying a liquid styling agent from one or more reservoirs to one or more of the anode-side housing, the cathode-side housing, or the absorbent pad, one or more fluid outlets for drawing the liquid styling agent back into the one or more reservoirs, a vacuum source coupled to the one or more fluid outlets, the vacuum source being configured to remove liquid styling agent that is unabsorbed by the pad, and a power module for supplying electrical power to the anode and/or the cathode to thereby form an electrical potential difference between the anode and the cathode. In an open configuration, the anode-side housing and the cathode-side housing are spaced apart. In a closed configuration, at least portions of the anode-side housing and the cathode-side housing are in direct contact to enclose therewithin an electrolysis zone, whereby hair within the electrolysis zone contacts the styling agent and is styled via electrolysis resulting at least in part from the potential difference.

Embodiments of the invention may include one or more of the following in any of a variety of combinations. The membrane may be absorbent, electrolyte-permeable, ion-conductive, and/or ion-selective. The device may include an anode-side foam seal disposed along the outer perimeter of the anode-side housing, and/or a cathode-side foam seal disposed along the outer perimeter of the cathode-side housing. The device may include an anode-side vacuum line fluidly connecting the anode-side foam seal to a second vacuum source, and/or a cathode-side vacuum line fluidly connecting the cathode-side foam seal to the second vacuum source. The vacuum source and the second vacuum source may be the same vacuum source or different vacuum sources. The anode-side foam seal and/or the cathode-side foam seal may include, consist essentially of, or consist of, at least in part, an open-cell foam. The device may include a first solid seal disposed along an outer edge of the anode-side foam seal to prevent lateral flow of styling agent from the anode-side foam seal. The device may include a second solid seal disposed along an outer edge of the cathode-side foam seal to prevent lateral flow of styling agent from the cathode-side foam seal.

The device may include a foam seal disposed along the outer perimeter of the anode-side housing or along the outer perimeter of the cathode-side housing. The device may include a vacuum line fluidly connecting the foam seal to a second vacuum source. The foam seal may include, consist essentially of, or consist of, at least in part, an open-cell foam. The device may include a solid seal disposed along an outer edge of the foam seal to prevent lateral flow of styling agent from the foam seal.

The device may include a control system for controlling the power module and flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on (i) an ionic conductivity between the anode and the cathode and/or (ii) a temperature of the styling agent. The control system may be configured to reverse a polarity of the potential difference formed by the power module. The device may include a hinge connecting the anode-side housing and the cathode-side housing. The device may include a handle extending from the hinge. The anode-side housing and/or the cathode-side housing may rotate at the hinge to transition between the open configuration and the closed configuration.

The styling agent may include, consist essentially of, or consist of an electrolyte. The styling agent may include, consist essentially of, or consist of a solution of water and one or more salts. The one or more salts may include, consist essentially of, or consist of one or more of sodium bicarbonate, sodium chloride, sodium carbonate, sodium sulfate, sodium iodide, magnesium sulfate, magnesium chloride, calcium chloride, potassium chloride, potassium iodide, potassium sulfate, potassium bicarbonate, potassium carbonate, sodium acetate, sodium maleate, lithium sulfate, sodium citrate, sodium phosphate, chromium chloride, aluminum chloride, or Epsom salt. The styling agent may include, consist essentially of, or consist of an organic acid and/or a salt of an organic acid.

In another aspect, embodiments of the invention feature a hairstyling device that includes, consists essentially of, or consists of a first housing having an outer perimeter, a second housing having an outer perimeter, one or more fluid inlets for supplying a liquid styling agent to at least one of the first housing or the second housing, fluidly connected to the one or more fluid inlets, a supply chamber containing therewithin first and second electrically conductive electrodes, and a power module for supplying electrical power to the first electrode and/or the second electrode to thereby form an electrical potential difference between the first electrode and the second electrode, whereby the styling agent undergoes electrolysis, resulting at least in part from the potential difference, before being supplied to the one or more fluid inlets. In an open configuration, the first housing and the second housing are spaced apart. In a closed configuration, at least portions of the first housing and the second housing are in direct contact to enclose therewithin a styling zone, whereby hair within the styling zone contacts the styling agent and is styled thereby.

Embodiments of the invention may include one or more of the following in any of a variety of combinations. The first electrode may be a cathode and the second electrode may be an anode, or vice versa. The device may include a foam seal disposed along the outer perimeter of the first housing. The device may include a vacuum line fluidly connecting the foam seal to a vacuum source. The foam seal may include, consist essentially of, or consist of, at least in part, an open-cell foam. The device may include a solid seal disposed along an outer edge of the foam seal to prevent lateral flow of styling agent from the foam seal. The device may include a second foam seal disposed along the outer perimeter of the second housing. At least a portion of the second foam seal may contact the foam seal in the closed configuration. The second seal may include, consist essentially of, or consist of, at least in part, an open-cell foam. The device may include a second solid seal disposed along an outer edge of the second foam seal to prevent lateral flow of styling agent from the second foam seal.

The device may include a control system for controlling the power module and flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on (i) an ionic conductivity of the styling agent and/or (ii) a temperature of the styling agent. The control system may be configured to reverse a polarity of the potential difference formed by the power module.

The device may include one or more of a membrane disposed within the first housing or the second housing, an absorbent pad disposed adjacent to the membrane, one or more fluid outlets for drawing the liquid styling agent back out of the first housing, the second housing, or both, and a vacuum source coupled to the one or more fluid outlets, the vacuum source being configured to remove liquid styling agent that is unabsorbed by the pad. The membrane may be absorbent, electrolyte-permeable, ion-conductive, and/or ion-selective. The device may include a hinge connecting the first housing and the second housing. The device may include a handle extending from the hinge. The first housing and/or the second housing may rotate at the hinge to transition between the open configuration and the closed configuration.

The styling agent may include, consist essentially of, or consist of an electrolyte. The styling agent may include, consist essentially of, or consist of a solution of water and one or more salts. The one or more salts may include, consist essentially of, or consist of one or more of sodium bicarbonate, sodium chloride, sodium carbonate, sodium sulfate, sodium iodide, magnesium sulfate, magnesium chloride, calcium chloride, potassium chloride, potassium iodide, potassium sulfate, potassium bicarbonate, potassium carbonate, sodium acetate, sodium maleate, lithium sulfate, sodium citrate, sodium phosphate, chromium chloride, aluminum chloride, or Epsom salt. The styling agent may include, consist essentially of, or consist of an organic acid and/or a salt of an organic acid.

In yet another aspect, embodiments of the invention feature a hairstyling device that includes, consists essentially of, or consists of a first housing having an outer perimeter, a second housing having an outer perimeter, one or more fluid inlets for supplying a liquid styling agent to the first housing, the second housing, or both, one or more fluid outlets for drawing the liquid styling agent back out of the first housing, the second housing, or both, a vacuum source coupled to the one or more fluid outlets, and fluidly connected to the one or more fluid inlets, a supply chamber for containing the styling agent therewithin. In an open configuration, the first housing and the second housing are spaced apart. In a closed configuration, at least portions of the first housing and the second housing are in direct contact to enclose therewithin a styling zone, whereby hair within the styling zone contacts the styling agent and is styled thereby.

Embodiments of the invention may include one or more of the following in any of a variety of combinations. The device may include one or more membranes disposed within the first housing and/or the second housing. The device may include an absorbent pad disposed adjacent to the membrane(s). The membrane may be absorbent, electrolyte-permeable, ion-conductive, and/or ion-selective. The device may include a foam seal disposed along the outer perimeter of the first housing. The device may include a vacuum line fluidly connecting the foam seal to a second vacuum source. The foam seal may include, consist essentially of, or consist of, at least in part, an open-cell foam. The device may include a solid seal disposed along an outer edge of the foam seal to prevent lateral flow of styling agent from the foam seal. The device may include a second foam seal disposed along the outer perimeter of the second housing. At least a portion of the second foam seal may contact the foam seal in the closed configuration. The second seal may include, consist essentially of, or consist of, at least in part, an open-cell foam. The device may include a second solid seal disposed along an outer edge of the second foam seal to prevent lateral flow of styling agent from the second foam seal.

The device may include a control system for controlling flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on (i) an ionic conductivity of the styling agent and/or (ii) a temperature of the styling agent. The device may include a hinge connecting the first housing and the second housing. The device may include a handle extending from the hinge. The first housing and/or the second housing may rotate at the hinge to transition between the open configuration and the closed configuration.

The styling agent may include, consist essentially of, or consist of an alkaline solution. The device may include, fluidly connected to the one or more fluid inlets, a second supply chamber for containing a neutralizing agent therewithin. The neutralizing agent may include, consist essentially of, or consist of an acidic solution.

In another aspect, embodiments of the invention feature a method of styling hair. A hairstyling device is provided. The hairstyling device includes, consists essentially of, or consists of (i) a first housing having an outer perimeter and (ii) a second housing having an outer perimeter. In an open configuration, the first housing and the second housing are spaced apart. In a closed configuration, at least portions of the first housing and the second housing are in direct contact to enclose therewithin a styling zone. A hair bundle is enclosed at least partially within the styling zone. An alkaline solution is supplied within the styling zone. The hair bundle is styled by contacting the hair bundle with the alkaline solution, and moving the device along a length of the hair bundle. Thereafter, an acidic solution is supplied within the styling zone. Alkaline solution remaining on the hair bundle is neutralized by contacting the hair bundle with the acidic solution, and moving the device along the length of the hair bundle.

Embodiments of the invention may include one or more of the following in any of a variety of combinations. Styling the hair bundle may include, consist essentially of, or consist of moving the device along the length of the hair bundle for a first plurality of strokes. Neutralizing alkaline solution remaining on the hair bundle may include, consist essentially of, or consist of moving the device along the length of the hair bundle for a second plurality of strokes. The first plurality of strokes may be equal to the second plurality of strokes. Styling the hair bundle may include, consist essentially of, or consist of moving the device along the length of the hair bundle for at least four strokes, or for at least ten strokes. Neutralizing alkaline solution remaining on the hair bundle may include, consist essentially of, or consist of moving the device along the length of the hair bundle for a plurality of strokes. Neutralizing alkaline solution remaining on the hair bundle may include, consist essentially of, or consist of moving the device along the length of the hair bundle for at least four strokes, or for at least ten strokes.

Supplying the alkaline solution may include, consist essentially of, or consist of electrolyzing a styling agent via application of a first potential difference thereto. Supplying the acidic solution may include, consist essentially of, or consist of applying a second potential difference to the styling agent. The first and second potential differences may have opposite polarities. The styling agent may be electrolyzed within the first housing and/or the second housing. After and/or during styling the hair, leakage of the alkaline solution may be prevented with a first foam seal disposed along the outer perimeter of the first housing, a second foam seal disposed along the outer perimeter of the second housing, or both. The first foam seal and/or the second foam seal may include, consist essentially of, or consist of, at least in part, an open-cell foam. Preventing leakage of the alkaline solution may include, consist essentially of, or consist of removing alkaline solution from the first foam seal and/or the second foam seal with a vacuum source. Leakage of the alkaline solution may be prevented by removing excess alkaline solution from the first housing and/or the second housing with a vacuum source.

In yet another aspect, embodiments of the invention feature a method of coloring hair. A device is provided. The device includes, consists essentially of, or consists of (i) a first housing containing a first electrode, (ii) a second housing containing a second electrode, (iii) disposed over the second electrode, a first masking layer including, consisting essentially of, or consisting of an electrically insulating material and defining a window therethrough, and (iv) disposed over the first masking layer, a second masking layer including, consisting essentially of, or consisting of an absorbent material. In an open configuration, the first housing and the second housing are spaced apart. In a closed configuration, at least portions of the first housing and the second housing are in direct contact to enclose therewithin a treatment zone. A hair bundle is at least partially enclosed within the treatment zone. A bleaching electrolyte is supplied within the treatment zone. The hair bundle is pre-treated by contacting the hair bundle with the bleaching electrolyte, and moving the device along a length of the hair bundle. Thereafter, a coloring electrolyte is supplied within the treatment zone. The hair bundle is colored by contacting the hair bundle with the coloring electrolyte, and moving the device along the length of the hair bundle.

Embodiments of the invention may include one or more of the following in any of a variety of combinations. After coloring the hair bundle, coloring electrolyte remaining on the hair bundle may be neutralized by (i) supplying an acidic solution within the treatment zone, and (ii) contacting the hair bundle with the acidic solution, and moving the device along the length of the hair bundle. The hair bundle may be washed, before coloring the hair bundle, to remove bleaching electrolyte therefrom. The hair bundle may be washed, after coloring the hair bundle, to remove coloring electrolyte therefrom. During pre-treating and/or coloring the hair bundle, (i) an electrolyte pH may be at least 13 beneath and/or away from the second masking layer (e.g., at or proximate the second electrode) and/or (ii) the electrolyte pH may be less than 13 on the second masking layer.

After pre-treating the hair, leakage of the bleaching electrolyte may be prevented with a first foam seal disposed along an outer perimeter of the first housing, a second foam seal disposed along an outer perimeter of the second housing, or both. The first foam seal and/or the second foam seal may include, consist essentially of, or consist of, at least in part, an open-cell foam. Preventing leakage of the bleaching electrolyte may include, consist essentially of, or consist of removing bleaching electrolyte from the first foam seal and/or the second foam seal with a vacuum source. Leakage of the bleaching electrolyte may be prevented by removing excess bleaching electrolyte from the first housing and/or the second housing with a vacuum source.

In another aspect, embodiments of the invention feature a hair-coloring device that includes, consists essentially of, or consists of a first housing containing a first electrode, a second housing containing a second electrode, disposed on the second housing over the second electrode, a first masking layer comprising an electrically insulating material and defining a window therethrough, disposed over the first masking layer, a second masking layer comprising an absorbent material, one or more fluid inlets for supplying one or more liquid treatment agents to the first housing, the second housing, or both, one or more fluid outlets for drawing the one or more liquid treatment agents back out of the first housing, the second housing, or both, a vacuum source coupled to the one or more fluid outlets, and fluidly connected to the one or more fluid inlets, one or more supply chambers for containing the one or more liquid treatment agents therewithin. In an open configuration, the first housing and the second housing are spaced apart. In a closed configuration, at least portions of the first housing and the second housing are in direct contact to enclose therewithin a treatment zone, whereby hair within the treatment zone contacts one of the liquid treatment agents and is treated thereby.

Embodiments of the invention may include one or more of the following in any of a variety of combinations. The device may include a foam seal disposed along an outer perimeter of the first housing. At least a portion of the foam seal may contact the second housing in the closed configuration. The device may include a vacuum line fluidly connecting the foam seal to a vacuum source. The foam seal may include, consist essentially of, or consist of, at least in part, an open-cell foam. The device may include a solid seal disposed along an outer edge of the foam seal to prevent lateral flow of liquid treatment agent from the foam seal. The device may include a second foam seal disposed along an outer perimeter of the second housing. At least a portion of the second foam seal may contact the foam seal in the closed configuration. The second foam seal may include, consist essentially of, or consist of, at least in part, an open-cell foam. The device may include a second solid seal disposed along an outer edge of the second foam seal to prevent lateral flow of liquid treatment agent from the second foam seal. The one or more liquid treatment agents may include, consist essentially of, or consist of a bleaching electrolyte, a coloring electrolyte, and/or an acidic neutralizing agent.

In an aspect, embodiments of the invention feature a hairstyling device that includes, consists essentially of, or consists of an anode-side housing having an outer perimeter, an electrically conductive anode disposed within the anode-side housing, a membrane disposed over the anode, an anode-side foam seal disposed along the outer perimeter of the anode-side housing, a cathode-side housing having an outer perimeter, an electrically conductive cathode disposed within the cathode-side housing, a cathode-side foam seal disposed along the outer perimeter of the cathode-side housing, one or more fluid inlets for supplying a liquid styling agent from one or more reservoirs to the anode-side housing, the cathode-side housing, or both, one or more fluid outlets for drawing the liquid styling agent back into the one or more reservoirs, and a power module for supplying electrical power to the anode and/or the cathode to thereby form an electrical potential difference between the anode and the cathode. In an open configuration, the anode-side foam seal and the cathode-side foam seal are spaced apart. In a closed configuration, at least portions of the anode-side foam seal and the cathode-side foam seal are in direct contact to enclose therewithin an electrolysis zone, whereby hair within the electrolysis zone contacts the styling agent and is styled via electrolysis resulting at least in part from the potential difference.

Embodiments of the invention may include one or more of the following in any of a variety of combinations. The membrane may be absorbent, electrolyte-permeable, ion-conductive, and/or ion-selective. The device may include an anode-side vacuum line fluidly connecting the anode-side foam seal to a first vacuum source, and/or a cathode-side vacuum line fluidly connecting the cathode-side foam seal to a second vacuum source. The first vacuum source and the second vacuum source may be the same vacuum source or different vacuum sources. The device may include a control system for controlling the power module and flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on an ionic conductivity between the anode and the cathode. The control system may be configured to reverse a polarity of the potential difference formed by the power module.

The device may include a hinge connecting the anode-side housing and the cathode-side housing. The device may include a handle extending from the hinge. The anode-side housing and/or the cathode-side housing may rotate at the hinge to transition between the open configuration and the closed configuration. The anode-side foam seal and/or the cathode-side foam seal may include, consist essentially of, or consist of, at least in part, an open-cell foam. The device may include a first solid seal disposed along an outer edge of the anode-side foam seal to prevent lateral flow of styling agent from the anode-side foam seal. The device may include a second solid seal disposed along an outer edge of the cathode-side foam seal to prevent lateral flow of styling agent from the cathode-side foam seal.

The styling agent may include, consist essentially of, or consist of an electrolyte. The styling agent may include, consist essentially of, or consist of a solution of water and one or more salts. The one or more salts may include, consist essentially of, or consist of one or more of sodium bicarbonate, sodium chloride, sodium carbonate, sodium sulfate, sodium iodide, magnesium sulfate, magnesium chloride, calcium chloride, potassium chloride, potassium iodide, potassium sulfate, potassium bicarbonate, potassium carbonate, sodium acetate, sodium maleate, lithium sulfate, sodium citrate, sodium phosphate, chromium chloride, aluminum chloride, or Epsom salt. The styling agent may include, consist essentially of, or consist of an organic acid and/or a salt of an organic acid.

In another aspect, embodiments of the invention feature a hairstyling device that includes, consists essentially of, or consists of a first housing having an outer perimeter, a first electrically conductive electrode disposed within the first housing, a foam seal disposed along the outer perimeter of the first housing, a second housing having an outer perimeter, a second electrically conductive electrode disposed within the second housing, one or more membranes disposed over the first electrode and/or the second electrode, one or more fluid inlets for supplying a liquid styling agent from one or more reservoirs to the first housing, the second housing, or both, one or more fluid outlets for drawing the liquid styling agent back into the one or more reservoirs, and a power module for supplying electrical power to the first electrode and/or the second electrode to thereby form an electrical potential difference between the first electrode and the second electrode. In an open configuration, the foam seal and the second housing are spaced apart from each other. In a closed configuration, at least portions of the foam seal and the second housing are in direct contact to enclose therewithin an electrolysis zone, whereby hair within the electrolysis zone contacts the styling agent and is styled via electrolysis resulting at least in part from the potential difference.

Embodiments of the invention may include one or more of the following in any of a variety of combinations. The first electrode may be a cathode and the second electrode may be an anode, or vice versa. The membrane may be absorbent, electrolyte-permeable, ion-conductive, and/or ion-selective. The device may include a vacuum line fluidly connecting the foam seal to a vacuum source. The device may include a control system for controlling the power module and flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on an ionic conductivity between the first electrode and the second electrode. The control system may be configured to reverse a polarity of the potential difference formed by the power module.

The device may include a hinge connecting the first housing and the second housing. The device may include a handle extending from the hinge. The first housing and/or the second housing may rotate at the hinge to transition between the open configuration and the closed configuration. The foam seal may include, consist essentially of, or consist of, at least in part, an open-cell foam. The device may include a solid seal disposed along an outer edge of the foam seal to prevent lateral flow of styling agent from the foam seal.

The styling agent may include, consist essentially of, or consist of an electrolyte. The styling agent may include, consist essentially of, or consist of a solution of water and one or more salts. The one or more salts may include, consist essentially of, or consist of one or more of sodium bicarbonate, sodium chloride, sodium carbonate, sodium sulfate, sodium iodide, magnesium sulfate, magnesium chloride, calcium chloride, potassium chloride, potassium iodide, potassium sulfate, potassium bicarbonate, potassium carbonate, sodium acetate, sodium maleate, lithium sulfate, sodium citrate, sodium phosphate, chromium chloride, aluminum chloride, or Epsom salt. The styling agent may include, consist essentially of, or consist of an organic acid and/or a salt of an organic acid.

In yet another aspect, embodiments of the invention feature a hairstyling device that includes, consists essentially of, or consists of a first housing having an outer perimeter, a foam seal disposed along the outer perimeter of the first housing, a second housing having an outer perimeter, one or more membranes disposed within the first housing and/or the second housing, one or more fluid inlets for supplying a liquid styling agent to the first housing, the second housing, or both, one or more fluid outlets for drawing the liquid styling agent back out of the first housing, the second housing, or both, fluidly connected to the one or more fluid inlets, a supply chamber containing therewithin first and second electrically conductive electrodes, and a power module for supplying electrical power to the first electrode and/or the second electrode to thereby form an electrical potential difference between the first electrode and the second electrode, whereby the styling agent undergoes electrolysis, resulting at least in part from the potential difference, before being supplied to the one or more fluid inlets. In an open configuration, the foam seal and the second housing are spaced apart. In a closed configuration, at least portions of the foam seal and the second housing are in direct contact to enclose therewithin a styling zone, whereby hair within the styling zone contacts the styling agent and is styled thereby.

Embodiments of the invention may include one or more of the following in any of a variety of combinations. The first electrode may be a cathode and the second electrode may be an anode, or vice versa. The membrane may be absorbent, electrolyte-permeable, ion-conductive, and/or ion-selective. The device may include a vacuum line fluidly connecting the foam seal to a vacuum source. The device may include a control system for controlling the power module and flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on an ionic conductivity of the styling agent. The control system may be configured to reverse a polarity of the potential difference formed by the power module.

The device may include a hinge connecting the first housing and the second housing. The device may include a handle extending from the hinge. The first housing and/or the second housing may rotate at the hinge to transition between the open configuration and the closed configuration. The foam seal may include, consist essentially of, or consist of, at least in part, an open-cell foam. The device may include a solid seal disposed along an outer edge of the foam seal to prevent lateral flow of styling agent from the foam seal.

The device may include a second foam seal disposed along the outer perimeter of the second housing. At least a portion of the second foam seal may contact the foam seal in the closed configuration. The second seal may include, consist essentially of, or consist of, at least in part, an open-cell foam. The device may include a second solid seal disposed along an outer edge of the second foam seal to prevent lateral flow of styling agent from the second foam seal.

The styling agent may include, consist essentially of, or consist of an electrolyte. The styling agent may include, consist essentially of, or consist of a solution of water and one or more salts. The one or more salts may include, consist essentially of, or consist of one or more of sodium bicarbonate, sodium chloride, sodium carbonate, sodium sulfate, sodium iodide, magnesium sulfate, magnesium chloride, calcium chloride, potassium chloride, potassium iodide, potassium sulfate, potassium bicarbonate, potassium carbonate, sodium acetate, sodium maleate, lithium sulfate, sodium citrate, sodium phosphate, chromium chloride, aluminum chloride, or Epsom salt. The styling agent may include, consist essentially of, or consist of an organic acid and/or a salt of an organic acid.

In another aspect, embodiments of the invention feature a hairstyling device that includes, consists essentially of, or consists of a first housing having an outer perimeter, a foam seal disposed along the outer perimeter of the first housing, a second housing having an outer perimeter, one or more fluid inlets for supplying a liquid styling agent to the first housing, the second housing, or both, one or more fluid outlets for drawing the liquid styling agent back out of the first housing, the second housing, or both, and fluidly connected to the one or more fluid inlets, a supply chamber for containing the styling agent therewithin. In an open configuration, the foam seal and the second housing are spaced apart. In a closed configuration, at least portions of the foam seal and the second housing are in direct contact to enclose therewithin a styling zone, whereby hair within the styling zone contacts the styling agent and is styled thereby.

Embodiments of the invention may include one or more of the following in any of a variety of combinations. The device may include one or more membranes disposed within the first housing and/or the second housing. The membrane may be absorbent, electrolyte-permeable, ion-conductive, and/or ion-selective. The device may include a vacuum line fluidly connecting the foam seal to a vacuum source. The device may include a control system for controlling flow of the styling agent. The device may include one or more sensors for detecting when the hairstyling device is in the open configuration and/or in the closed configuration. The one or more sensors may include, consist essentially of, or consist of one or more switches, one or more Hall effect sensors, and/or one or more pressure sensors. The control system may be configured to supply the styling agent only when the hairstyling device is in the closed configuration. The control system may be configured to regulate flow of the styling agent based at least in part on an ionic conductivity of the styling agent.

The device may include a hinge connecting the first housing and the second housing. The device may include a handle extending from the hinge. The first housing and/or the second housing may rotate at the hinge to transition between the open configuration and the closed configuration. The foam seal may include, consist essentially of, or consist of, at least in part, an open-cell foam. The device may include a solid seal disposed along an outer edge of the foam seal to prevent lateral flow of styling agent from the foam seal.

The device may include a second foam seal disposed along the outer perimeter of the second housing. At least a portion of the second foam seal may contact the foam seal in the closed configuration. The second seal may include, consist essentially of, or consist of, at least in part, an open-cell foam. The device may include a second solid seal disposed along an outer edge of the second foam seal to prevent lateral flow of styling agent from the second foam seal.

The styling agent may include, consist essentially of, or consist of an alkaline solution. The device may include, fluidly connected to the one or more fluid inlets, a second supply chamber for containing a neutralizing agent therewithin. The neutralizing agent may include, consist essentially of, or consist of an acidic solution.

In yet another aspect, embodiments of the invention feature a method of styling hair. A hairstyling device is provided. The hairstyling device includes, consists essentially of, or consists of (i) a first housing having an outer perimeter and (ii) a second housing having an outer perimeter. In an open configuration, the first housing and the second housing are spaced apart. In a closed configuration, at least portions of the first housing and the second housing are in direct contact to enclose therewithin a styling zone. A hair bundle is enclosed at least partially within the styling zone. An alkaline solution is supplied within the styling zone. The hair bundle is styled by contacting the hair bundle with the alkaline solution, and moving the device along a length of the hair bundle. Thereafter, an acidic solution is supplied within the styling zone. Alkaline solution remaining on the hair bundle is neutralized by contacting the hair bundle with the acidic solution, and moving the device along the length of the hair bundle.

Embodiments of the invention may include one or more of the following in any of a variety of combinations. Styling the hair bundle may include, consist essentially of, or consist of moving the device along the length of the hair bundle for a first plurality of strokes. Neutralizing alkaline solution remaining on the hair bundle may include, consist essentially of, or consist of moving the device along the length of the hair bundle for a second plurality of strokes. The first plurality of strokes may be equal to the second plurality of strokes. Styling the hair bundle may include, consist essentially of, or consist of moving the device along the length of the hair bundle for at least four strokes, or for at least ten strokes. Neutralizing alkaline solution remaining on the hair bundle may include, consist essentially of, or consist of moving the device along the length of the hair bundle for a plurality of strokes. Neutralizing alkaline solution remaining on the hair bundle may include, consist essentially of, or consist of moving the device along the length of the hair bundle for at least four strokes, or for at least ten strokes.

Supplying the alkaline solution may include, consist essentially of, or consist of electrolyzing a styling agent via application of a first potential difference thereto. Supplying the acidic solution may include, consist essentially of, or consist of applying a second potential difference to the styling agent. The first and second potential differences may have opposite polarities. The styling agent may be electrolyzed within the first housing and/or the second housing. After and/or during styling the hair, leakage of the alkaline solution may be prevented with a first foam seal disposed along the outer perimeter of the first housing, a second foam seal disposed along the outer perimeter of the second housing, or both. The first foam seal and/or the second foam seal may include, consist essentially of, or consist of, at least in part, an open-cell foam. Preventing leakage of the alkaline solution may include, consist essentially of, or consist of removing alkaline solution from the first foam seal and/or the second foam seal with a vacuum source.

In another aspect, embodiments of the invention feature a method of coloring hair. A device is provided. The device includes, consists essentially of, or consists of (i) a first housing containing a first electrode, (ii) a second housing containing a second electrode, (iii) disposed over the second electrode, a first masking layer including, consisting essentially of, or consisting of an electrically insulating material and defining a window therethrough, and (iv) disposed over the first masking layer, a second masking layer including, consisting essentially of, or consisting of an absorbent material. In an open configuration, the first housing and the second housing are spaced apart. In a closed configuration, at least portions of the first housing and the second housing are in direct contact to enclose therewithin a treatment zone. A hair bundle is at least partially enclosed within the treatment zone. A bleaching electrolyte is supplied within the treatment zone. The hair bundle is pre-treated by contacting the hair bundle with the bleaching electrolyte, and moving the device along a length of the hair bundle. Thereafter, a coloring electrolyte is supplied within the treatment zone. The hair bundle is colored by contacting the hair bundle with the coloring electrolyte, and moving the device along the length of the hair bundle.

Embodiments of the invention may include one or more of the following in any of a variety of combinations. After coloring the hair bundle, coloring electrolyte remaining on the hair bundle may be neutralized by (i) supplying an acidic solution within the treatment zone, and (ii) contacting the hair bundle with the acidic solution, and moving the device along the length of the hair bundle. The hair bundle may be washed, before coloring the hair bundle, to remove bleaching electrolyte therefrom. The hair bundle may be washed, after coloring the hair bundle, to remove coloring electrolyte therefrom. During pre-treating and/or coloring the hair bundle, (i) an electrolyte pH may be at least 13 beneath the second masking layer and/or (ii) the electrolyte pH may be less than 13 on the second masking layer.

After pre-treating the hair, leakage of the bleaching electrolyte may be prevented with a first foam seal disposed along an outer perimeter of the first housing, a second foam seal disposed along an outer perimeter of the second housing, or both. The first foam seal and/or the second foam seal may include, consist essentially of, or consist of, at least in part, an open-cell foam. Preventing leakage of the bleaching electrolyte may include, consist essentially of, or consist of removing bleaching electrolyte from the first foam seal and/or the second foam seal with a vacuum source.

In yet another aspect, embodiments of the invention feature a hair-coloring device that includes, consists essentially of, or consists of a first housing containing a first electrode, a second housing containing a second electrode, disposed on the second housing over the second electrode, a first masking layer comprising an electrically insulating material and defining a window therethrough, disposed over the first masking layer, a second masking layer comprising an absorbent material, one or more fluid inlets for supplying one or more liquid treatment agents to the first housing, the second housing, or both, one or more fluid outlets for drawing the one or more liquid treatment agents back out of the first housing, the second housing, or both, and fluidly connected to the one or more fluid inlets, one or more supply chambers for containing the one or more liquid treatment agents therewithin. In an open configuration, the first housing and the second housing are spaced apart. In a closed configuration, at least portions of the first housing and the second housing are in direct contact to enclose therewithin a treatment zone, whereby hair within the treatment zone contacts one of the liquid treatment agents and is treated thereby.

Embodiments of the invention may include one or more of the following in any of a variety of combinations. The device may include a foam seal disposed along an outer perimeter of the first housing. At least a portion of the foam seal may contact the second housing in the closed configuration. The device may include a vacuum line fluidly connecting the foam seal to a vacuum source. The foam seal may include, consist essentially of, or consist of, at least in part, an open-cell foam. The device may include a solid seal disposed along an outer edge of the foam seal to prevent lateral flow of liquid treatment agent from the foam seal. The device may include a second foam seal disposed along an outer perimeter of the second housing. At least a portion of the second foam seal may contact the foam seal in the closed configuration. The second foam seal may include, consist essentially of, or consist of, at least in part, an open-cell foam. The device may include a second solid seal disposed along an outer edge of the second foam seal to prevent lateral flow of liquid treatment agent from the second foam seal. The one or more liquid treatment agents may include, consist essentially of, or consist of a bleaching electrolyte, a coloring electrolyte, and/or an acidic neutralizing agent.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “about,” “substantially,” and “approximately” mean±10%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS. 1A-1E are schematic depictions of a hairstyling device and various leakage modalities in accordance with embodiments of the present invention;

FIG. 2 is a cross-sectional schematic of a portion of a handpiece in accordance with embodiments of the present invention;

FIG. 3A is an exploded view of the anode side of the handpiece of a hairstyling device in accordance with embodiments of the present invention;

FIG. 3B is an exploded view of the cathode side of the handpiece of a hairstyling device in accordance with embodiments of the present invention;

FIG. 3C is a cutaway view of the handpiece of FIGS. 3A and 3B with both the cathode and anode sides, clamped together as during operation, in accordance with embodiments of the present invention;

FIG. 3D is an image of an exemplary hairstyling device in accordance with embodiments of the present invention;

FIG. 3E is an image of an exemplary hairstyling device in accordance with embodiments of the present invention;

FIG. 4 is a schematic diagram of portions of a fluid management system that may be utilized in accordance with embodiments of the present invention;

FIG. 5A is a plan-view schematic of a cathode of a hairstyling device in accordance with embodiments of the present invention;

FIGS. 5B and 5C are plan-view schematics of the layers of a two-layer mask that may be applied to the cathode of FIG. 5A in accordance with embodiments of the present invention; and

FIG. 6 is a depiction of hair strands successfully colored with different colored dyes in accordance with embodiments of the present invention.

### DETAILED DESCRIPTION

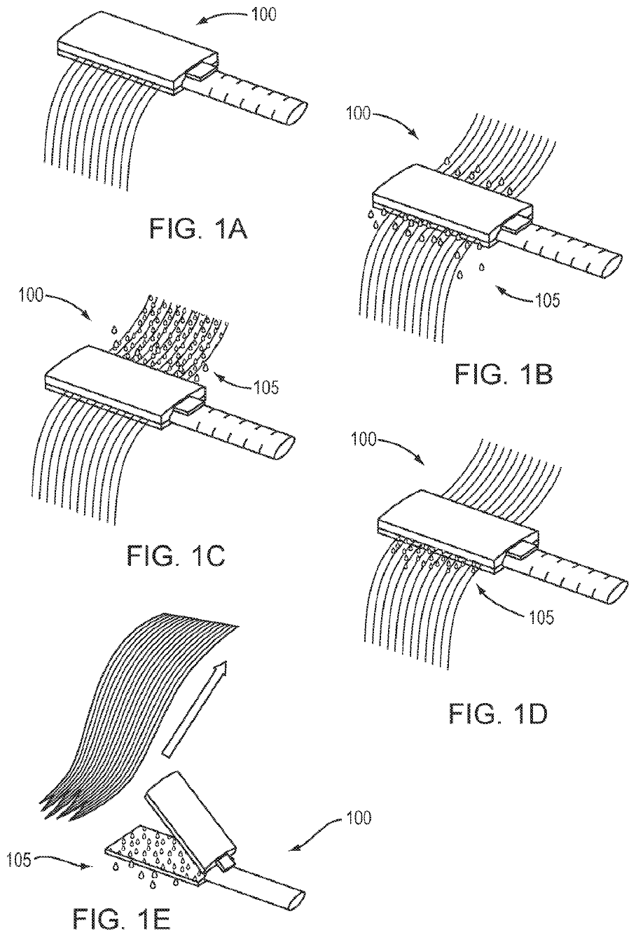

FIG. 1A depicts an exemplary device 100 in accordance with embodiments of the invention during hair styling. As shown, hair is clamped within the device 100 between the two electrodes, electrolysis is initiated, and the device 100 is moved down the length of the hair for styling thereof. Embodiments of the invention address leakage of the liquid styling agent from the styling device 100 that could potentially occur during various phases of operation. For example, FIG. 1B depicts an exemplary leakage mode, in which the styling agent 105 leaks from the device 100 due to over-pumping of the agent 105 into the handpiece (or the electrolysis zone), and/or lack of liquid sealing or scavenging during styling. FIG. 1C depicts another exemplary leakage mode, in which the styling agent 105 is deposited on the trailing edge of the hair as it exits the device 100. FIG. 1D depicts a similar leakage mode, in which excessive pressure is applied to hair saturated with the styling agent 105, leading to dripping of the styling agent 105 from the leading edge of the hair. Finally, FIG. 1E depicts leakage that may occur when the handpiece is repositioned to style another bundle of hair. As shown, the handpiece is typically opened after styling of the initial bundle of hair and repositioned to sandwich the next bundle of hair between the electrodes, resulting in potential leakage of the styling agent 105 from the electrolysis zone.

Various embodiments of the invention enable hair styling via by application of an alkaline solution, which can be produced by electrolysis, while reducing or substantially eliminating leakage of the liquid styling agent from the handpiece. In various embodiments, the inner perimeter of the handpiece incorporates an open cell foam barrier that absorbs excess liquid to prevent leakage. In various embodiments, this barrier is in fluid connection with a vacuum line that draws the liquid styling agent from the hair, through the foam, and back to one or more holding tanks. In addition, various embodiments of the invention include an optional solid seal on the outer edge of the foam to prevent flow of the styling agent through the outer edge of the foam. For example, the solid seal may surround the outer edge of the foam seal and, in various embodiments, may include, consist essentially of, or consist of rubber or another polymeric material. The solid seal may not be absorbent.

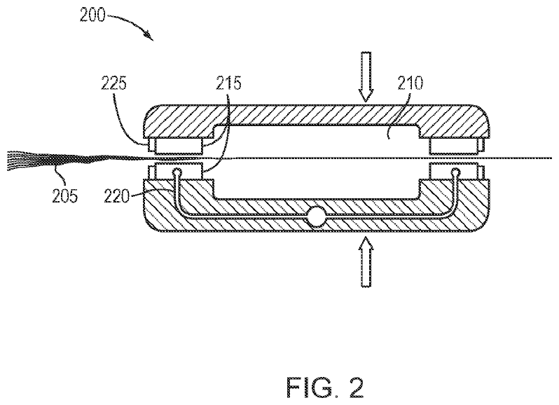

FIG. 2 is a cross-sectional schematic of a portion of a handpiece 200 in accordance with embodiments of the invention, depicting anti-leakage technology incorporated therewithin. In FIG. 2, opposing portions of the device (which may contain the electrodes) are clamped around a bundle of hair 205, and the handpiece is moving from right to left to style the hair introduced into the center electrolysis zone 210. As shown in FIG. 2, foam seals 215 may be disposed on each side (e.g., the anode side and the cathode side) of the handpiece 200, such that, when the handpiece 200 is in a closed configuration for styling hair, the foam seals 215 contact each other, sandwiching the hair 205 between them to prevent leakage of the styling agent from the hair. In other embodiments, the foam seal 215 is present only on one side of the handpiece 200 or the other, and the foam seal 215 contacts the opposite side when the handpiece is closed. For example, the side without the foam seal 215 may have a non-porous (e.g., plastic) surface (which may protrude from the side of the handpiece) positioned and configured to contact the foam seal 215 when the handpiece is closed. In such embodiments, only the single foam seal 215 need be in fluid connection with a vacuum line (or vacuum source) 220. As also shown, the device 200 may include one or more solid seals 225 on the outer edge of the foam 215 to prevent flow of the styling agent through the outer edge of the foam 215.

In various embodiments of the invention, the device may lack both foam seals 215 and may utilize the vacuum source 220 for leakage prevention. For example, the vacuum line 220 may be fluidly connected to the electrolysis zone 210 itself, and it may exhaust styling agent therefrom during styling of the hair. This prevents undue build-up of the styling agent within the device; sufficient styling agent remains in the electrolysis zone (e.g., absorbed by a porous membrane, substrate, or pad), while excess styling agent is removed from the device via vacuum. In various embodiments, the vacuum source may be a vacuum pump or a liquid pump. For example, one liquid pump may supply the styling agent into the electrolysis zone while a second liquid pump removes styling agent (along with air) from the device to prevent leakage. Such embodiments may feature, but do not require, foam seals, thereby enabling the device to be more compact and more completely clamp onto each hair bundle being styled.

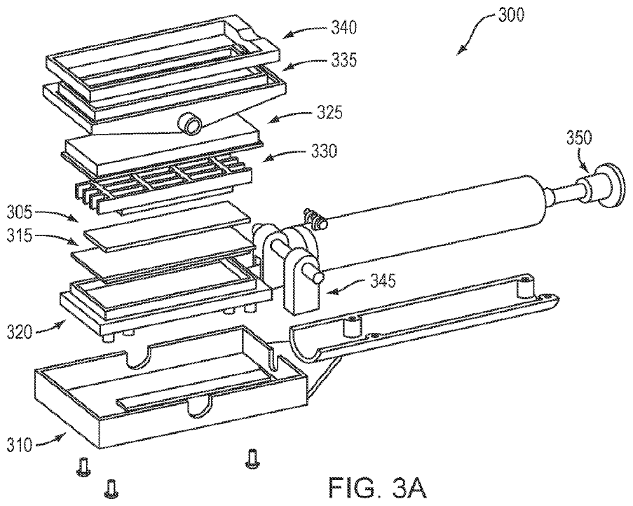

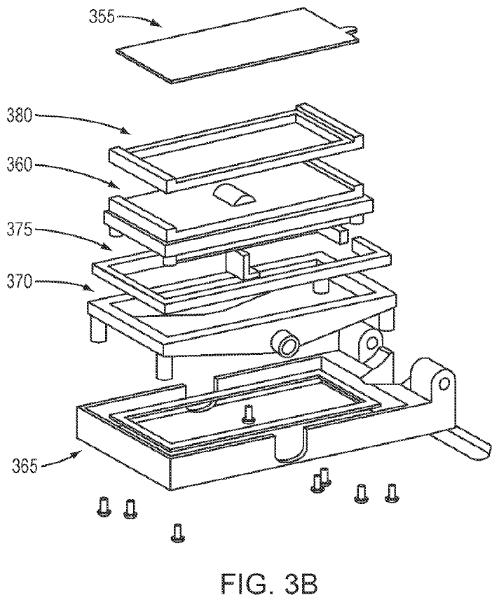

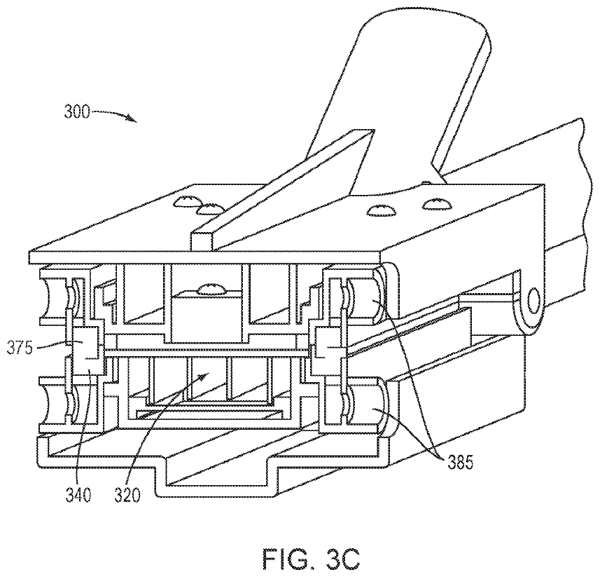

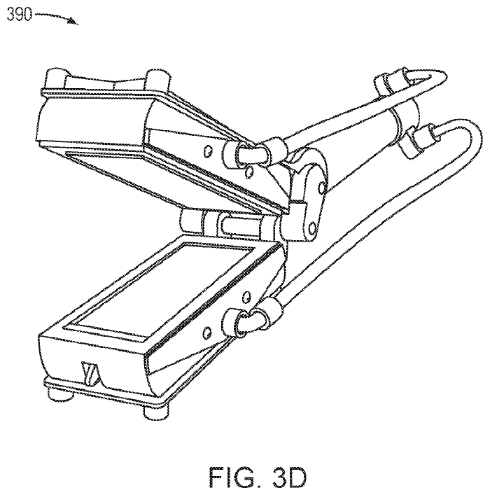

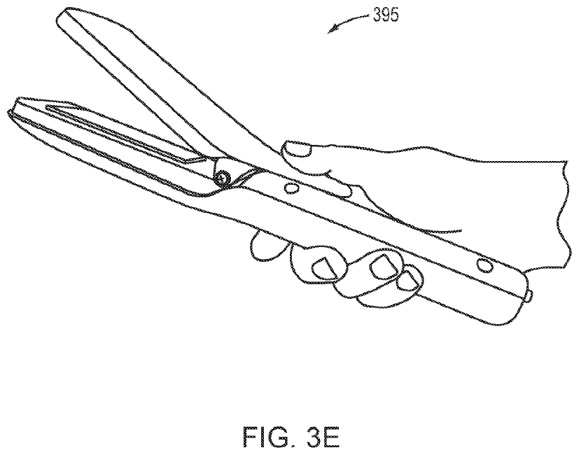

FIG. 3A is an exploded view of the anode side of the handpiece of a hairstyling device 300 in accordance with embodiments of the present invention. As shown, the anode 305 fits within the baseplate 310 of the anode side with a flow spacer 315 and flow box 320 therebetween. The anode 305 may include, consist essentially of, or consist of, for example, an electrically conductive plate. An absorbent, liquid-permeable, ion-conductive, and/or ion-selective (for example, a sulfonated tetrafluoroethylene-based fluoropolymer-copolymer such as NAFION) membrane 325 is disposed over the anode 305 with an offset spacer 330 therebetween. A sealing manifold 335 sits atop the membrane, and an optional sealing gasket 340 (e.g., an open-cell foam) fits within a recess defined in the sealing manifold 335. During operation, the liquid styling agent is pumped into the space over the anode 305 and is contained over and/or within the absorbent membrane 325. In various embodiments, the foam gasket 340 prevents leakage of the styling agent during operation. The sealing manifold 335 contains one or more connections to a vacuum line utilized to scavenge styling agent (e.g., excess styling agent within and/or above the membrane 325, and/or styling agent absorbed by the foam gasket 340) back to one or more holding tanks, thereby preventing leakage. As also shown, the handpiece features a hinge 345 to which the opposing cathode side of the handpiece mechanically connects (see FIG. 3B). The device may also include an adjustment screw 350 (or other adjustment means), which may be adjusted to alter the clamping force applied by the device.

In various embodiments, two or more of the components of the anode side of the handpiece may be integrated together into a single part retaining the functionality of the components. For example, in various embodiments, the anode flow box 320 and the sealing manifold 335 depicted in FIG. 3A may be integrated together into a single component.

FIG. 3B is an exploded view of the cathode side of the handpiece of a hairstyling device 300 in accordance with embodiments of the invention. As shown, the cathode 355 is mounted on a cathode-side mount box 360 that fits within the cathode-side baseplate 365 and within another sealing manifold 370. The device may include an optional foam gasket 375 (e.g., open-cell foam) that fits within the sealing manifold 370. The cathode 355 may include, consist essentially of, or consist of, for example, an electrically conductive plate. A seal 380 (e.g., a backing foam and/or adhesive) may seal the cathode 355 to the mount box 360. The cathode-side sealing manifold 370 may also contain (and/or route therewithin) one or more connections to a vacuum line utilized to scavenge any excess styling agent absorbed by the foam gasket 375, and/or above the cathode 355, back to one or more holding tanks, thereby preventing leakage.

The cathode 355 and/or the anode 305 is electrically coupled to a power source (e.g., a direct-current (DC) power source) to thereby enable the formation of a potential difference between the cathode 355 and the anode 305. This region between the cathode 355 and the anode 305 (e.g., the membrane 325 over the anode 305), also termed the “electrolysis zone” or the “anode box”) is the region in which a bundle of hair comes into contact with the liquid styling agent and is styled via electrolysis, as also described in the '056 patent and the '1312 application. In various embodiments, two or more of the components of the cathode side of the handpiece may be integrated together into a single part retaining the functionality of the components. For example, in various embodiments, the cathode mount box 360 and the sealing manifold 370 depicted in FIG. 3B may be integrated together into a single component.

In embodiments featuring them, the anode-side and cathode-side foam gaskets (or “seals”) may include, consist essentially of, or consist of open-cell foam, i.e., foam with internal pores that are sufficiently interconnected to form fluid paths through the foam. (In contrast, a “closed-cell” foam contains pores that are not interconnected; liquids typically are not flowable through a closed-cell foam.) Open-cell foams in accordance with embodiments of the invention may include, consist essentially of, or consist of one or more polymeric and/or elastomeric materials. In embodiments in which the vacuum source is directly fluidly connected to the electrolysis zone itself, the foam seals may be composed of closed-cell foam or may be absent.

In various embodiments, the anode and/or the cathode may include, consist essentially of, or consist of a conductive material, for example, graphite, titanium, platinum, aluminum, copper, stainless steel, alloys, other metals, ceramics, any other suitable conductive material, and or a combination thereof. In some embodiments, the anode and/or the cathode may also be coated with a corrosion resistant material, for example, gold, platinum, aluminum, any other corrosion resistant material or combination thereof. The anode and/or the cathode may be substantially flat electrodes, for example, plate-type electrodes, or they may be sintered electrodes, foams, or other porous constructions. In some embodiments, the surface of the anode and/or the cathode may include features, for example, contours, ridges, or any other features configured to aid in styling the hair of a user. The anode and the cathode may be spaced apart by a distance that defines the electrolysis zone.

As shown in FIG. 3A, the device may include a membrane 325 on the anode side. (In various embodiments, the membrane 325 may instead be on the cathode side, or both the anode side and the cathode sides may include membranes.) The membrane 325 may include, consist essentially of, or consist of an absorbent pad, sponge, or other porous material that serves as a reservoir for the styling agent and may contain a volume of the styling agent for use during electrolysis. In various embodiments, the membrane 325 may include, consist essentially of, or consist of a thin porous material that separates the volume of styling agent and allows slow permeation of the styling agent through the membrane. For example, in various embodiments the membrane may be composed of multiple layers, e.g., an upper thin porous layer that enables flow of the styling agent therethrough, and, therebelow, an absorbent pad or sponge layer that absorbs the styling agent and acts as the reservoir thereof. As utilized herein, an absorbent pad or pad layer that is “disposed adjacent to” a membrane may be a component separate from the membrane or an absorbent portion of a unitary construct (e.g., a two-layer construct) that includes a membrane layer and a pad layer. Alternatively, all or a portion of the membrane may be of non-porous construction but include an ion-conductive or ion-selective material that allows the flow of ionic current through the membrane. The membrane may be positioned such that hair may be disposed adjacent to or on the membrane during use. As shown in FIG. 3A, the membrane may cover the anode and thereby prevent direct contact between the anode and the cathode, even when the handpiece is closed during use.

FIG. 3C is a cutaway view of the handpiece 300 with both the cathode and anode sides, clamped together as during operation. As shown, if present, the foam gaskets 340, 375 of the cathode and anode sides mechanically contact each other to form a foam seal around the electrolysis zone that may help prevent leakage of the styling agent out of the handpiece. Excess styling agent is scavenged via one or more vacuum lines 385 present in the device. As detailed above, the one or more vacuum lines may be fluidly coupled to, and may exhaust excess styling agent from, the foam seals (if present) and/or from the electrolysis zone itself.

A photograph of an exemplary handpiece 390 for a styling device in accordance with embodiments of the invention is shown in FIG. 3D. In various embodiments, one or both sides of the handpiece may include an indicator to communicate the status of the device to the operator. For example, one or more indicator lights may be disposed on one or both sides of the handpiece. FIG. 3E is a photograph of another exemplary handpiece 395 (without other components, for clarity) for a styling device in accordance with embodiments of the invention, depicting opening of the cathode and anode sides via thumb actuation of a lever to overcome spring force configured to close the device.

In various embodiments, the handpiece may include a button or other control mechanism, operable by the operator, which controls the flow of styling agent into the device, and/or the potential difference between the anode and cathode that forms the electrolysis zone.

During operation, a bundle of hair passes through the handpiece between the cathode and anode sides. Force (e.g., spring force) may be applied to the hair via the clamping action of the handpiece. In an exemplary embodiment, as the hair passes vertically through the handpiece, the lower portions of the foam gasket wick in the styling agent that runs down the membrane in the electrolysis zone and the hair due to the force of gravity. The wicked styling agent is scavenged from the foam gaskets via the vacuum lines. In the electrolysis zone, the spring force of the handpiece holds the cathode in close proximity to, or even in direct contact with, the hair that is sliding upwards along the membrane on the anode side. In various embodiments, some styling agent may travel with the hair upward along the membrane via surface tension or pressure-driven flow from agent flowing through the membrane from the anode compartment or from agent being directly injected on the cathode side of the membrane, while additional styling agent may run downward along the membrane due to the force of gravity. Much of the excess styling agent therefore ends up within the lower leading edge of the foam seal, where it is removed via vacuum. The upper, trailing edge of the foam seal wicks in excess styling agent that is drawn upwards with the hair during styling. In various embodiments, hair exiting the handpiece after styling is damp but insufficiently wet to drip due to the absorbing action of the foam seal. In various embodiments, the foam gaskets surround and enclose the entire electrolysis zone, enabling the handpiece to operate in any orientation and to be utilized by a left-handed or right-handed operator.

In embodiments in which the foam gaskets are not present, the vacuum source scavenges the excess styling agent directly away from the electrolysis zone while leaving sufficient styling agent in the absorbent membrane (or portion thereof) for hair styling.

In various embodiments of the invention, one or more portions of the handpiece that come into contact with hair are removable and replaceable, for example as a consumable cartridge that may be replaced after the handpiece has been utilized for styling the hair of a first client and before styling a second client's hair. For example, one or more of the components shown in FIGS. 3A and 3B (including, e.g., the foam gasket(s), membrane, and one or more manifolds and/or spacers) may be replaceable as a separate component that is inserted into the handpiece before the styling of a new client's hair. Such embodiments enable more hygienic hair styling for each client. After each client's hair is styled, the used cartridge may be discarded and a new cartridge installed into the handpiece.

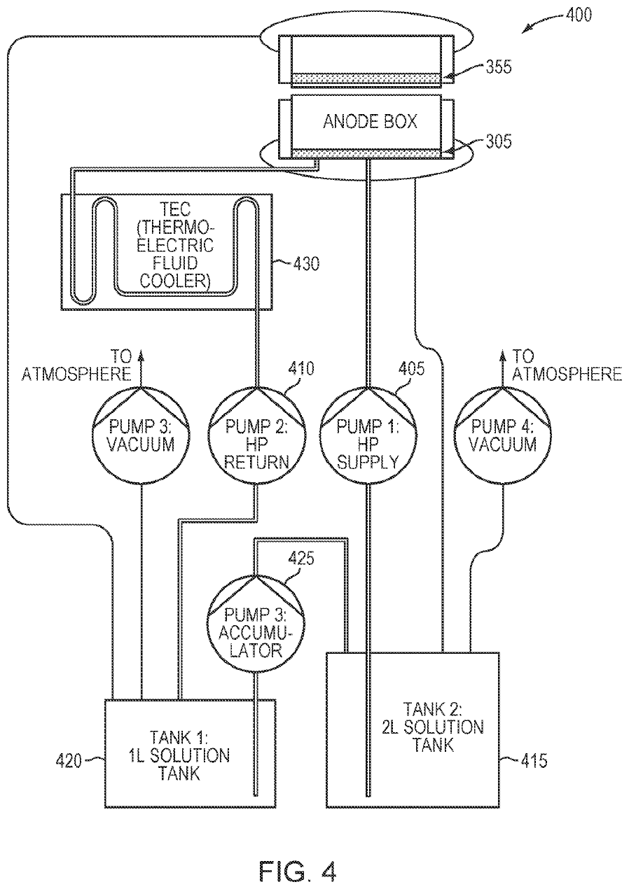

FIG. 4 is a schematic diagram of portions of a fluid management system 400 utilized in various embodiments of the present invention. As shown, pumps 405, 410 supply the styling agent, or “solution” to the handpiece (e.g., to the electrolysis zone) from a first holding tank 415 and return the styling agent back to a second holding tank 420. An accumulator pump 425 may control the flow of the styling agent between the two holding tanks 415, 420. In various embodiments, if styling agent is scavenged from the foam gaskets of the device, then additional pumps may be utilized to remove the styling agent from the gaskets and deliver it to the holding tank 420.

As detailed above, excess styling agent from the handpiece is drawn away via pump 410 back into the holding tanks 415, 420, from which the excess styling agent may be reused. As such, the pump 410 may be considered to be the “vacuum source” utilized to remove the excess styling agent from the device. In various embodiments during operation of the device, the supply pump 405 will draw styling agent from the holding tank 415 into the electrolysis zone of the handpiece. The return pump 410 then scavenges styling agent back from the anode box (e.g., at a bottom portion thereof). In various embodiments, the flow rate of the return pump 410 is different than that of the supply pump 405, and this differential flow rate causes the anode box to either fill, drain, or drive cross-membrane flow. For example, in an exemplary embodiment, the supply pump 405 may pump styling agent at approximately 150 ml/min while the return pump 410 pumps the styling agent away from the handpiece at a lower pumping rate, e.g., approximately 140 ml/min. This differential flow causes the anode box to fill until the styling agent is forced through the membrane in the anode side and into the hair clamped between the anode and cathode. As mentioned above, excess styling agent may be absorbed by the foam sealing gaskets during operation and returned to the holding tank via the vacuum pumps fluidly connected to the gaskets. In various embodiments, the various pumps are peristaltic pumps driven by motors (e.g., stepper motors). In various embodiments, the flow rates of the pumps 405, 410 may be controlled such that all “loose” styling agent within the anode box is removed from the device, and hair is styled by styling agent supplied by the absorbent membrane, thereby preventing leakage from the device. For example, the flow rate out of the handpiece (e.g., of styling agent and air) may be approximately equal to, or even greater than, the flow rate into the handpiece, so that substantially all of the styling agent present in the electrolysis zone is disposed within the absorbent membrane. In this manner, excess styling agent is scavenged away from the electrolysis zone, and leakage is prevented.

As shown in FIG. 4, in various embodiments the fluid management system 400 may include a fluid cooling system 430 that cools the styling agent. For example, the fluid cooling system 430 may include, consist essentially of, or consist of a thermoelectric cooler, a radiator, and/or an air cooler. As shown, the fluid cooling system 430 may be positioned to cool the fluid pumped out of the anode box by the return pump 410, as the temperature of the styling agent may increase during operation (e.g., due to the electrolysis in the anode box). Alternatively or in addition, the fluid cooling system 430 may be positioned to cool the fluid pumped to the anode box.

In various embodiments, after a bundle of hair has been styled via the handpiece, some of the styling agent (e.g., an alkaline liquid) may remain on and in contact with the bundle of hair. In various embodiments, after the bundle of hair has been styled, for example, straightened by application of an alkaline solution, a pH-neutralizing solution (e.g., an acidic liquid such as a citric acid solution) may be applied to the bundle of hair, via the handpiece, to neutralize the remnant styling agent. In other embodiments, the same electrolyte may be dispensed for both straightening and neutralizing, and the polarity of the electrodes in the handpiece is reversed for application of the neutralizing agent. That is, the styling agent may provide hydroxide ions that are applied to the bundle of hair for straightening, the electrode polarity may then be reversed, and the styling agent may then provide hydrogen ions to neutralize the alkaline styling agent remaining on the hair. In these embodiments, an electrolyte may be used that can support production of either alkaline or acidic solutions by electrolysis. Examples of such electrolytes include solutions of lithium-, sodium-, and/or potassium-based sulfate, sulfite, maleate, or nitrate salts. In various embodiments, the polarity of the electrodes may be controlled by a simple switch or button on the handpiece.

In various embodiments, the fluid management system 430 features a control system that senses the state of the device and controls the flow of styling agent accordingly. For example, when the handpiece is open (i.e., not clamping hair between the anode and cathode sides), hair is not being styled and no fluid is pumped by the system. The open or closed state of the handpiece may be detected via a switch and/or a sensor in the handpiece (e.g., a pressure sensor, a Hall effect sensor, etc.), etc. When the handpiece is closed and hair is to be treated, fluid is pumped via the differential flow rate of the supply and return pumps, as detailed above, causing the anode box to fill with styling agent. The styling agent wets the hair clamped in the device (e.g., via contact between the hair and the anode-side membrane) and ensures uniform ionic conductivity for electrolysis.

In various embodiments, the styling agent to be utilized for hair styling undergoes electrolysis within an electrolysis chamber or reservoir that is not within, but that is fluidly connected to, the handpiece. In such embodiments, the electrolyzed solution may be pumped to the handpiece for application to the hair after undergoing electrolysis. In such embodiments, the handpiece may lack one or both electrodes and may simply include one or more pads for application of the styling agent to hair within the handpiece. In such embodiments, excess styling agent may be removed directly from the electrolysis zone by the vacuum source, as detailed above. Moreover, such embodiments may lack the vacuum source, and even a fluid outlet from the handpiece. Since the solution undergoes electrolysis prior to delivery to the handpiece, it may be introduced into the handpiece, and the application of the solution to the hair provides sufficient “removal” of the solution from the handpiece, obviating the need for a dedicated removal outlet and system serving the handpiece.

In some embodiments, the flow of styling agent is controlled, at least in part, by the measured ionic conductivity between the anode and cathode such that the flow of styling agent is started or increased when low conductivity is detected. For example, in various embodiments, the electrolysis voltage may be provided by a constant-current power supply. The power supply may be configured to deliver a substantially constant current by modulating voltage (e.g., between 0 and 20 VDC). In various embodiments, the system voltage is automatically adjusted to achieve the desired current level (which may be, for example, approximately 5 amps). Accordingly, the voltage acts as a measure of electrical resistance (given the relationship between voltage and current), and, by proxy, a measure of styling agent presence between the anode and the cathode. Thus, in various embodiments of the invention, the fluid supply system operates in as a feedback control loop closed on voltages, thereby enabling optimal control of the flow of the styling agent based on an easily measurable parameter (i.e., the voltage between the anode and the cathode).

In various embodiments, the flow of styling agent is controlled, at least in part, via the measured temperature of the styling agent itself. Since the styling agent temperature may be altered as it undergoes electrolysis, the temperature of the styling agent may be measured (e.g., within the handpiece, and/or downstream from the handpiece in a component such as a fluid outlet or holding tank), and the flow of the styling agent controlled accordingly. Thus, various embodiments of the invention may feature one or more temperature sensors (e.g., thermocouples) in, on, and/or downstream of the handpiece.

In various embodiments, the control system may include a processor (e.g., a computer processor) for controlling the various system components and executing various styling procedures with the device is in use. The processor may be one or more general-purpose processors, but in other embodiments may utilize any of a wide variety of other technologies including special-purpose hardware, programmed micro-processor, micro-controller, peripheral integrated circuit element, a CSIC (Customer Specific Integrated Circuit), ASIC (Application Specific Integrated Circuit), a logic circuit, a digital signal processor, a programmable logic device such as an FPGA (Field Programmable Gate Array), PLD (Programmable Logic Device), PLC (Programmable Logic Controller), PLA (Programmable Logic Array), RFID processor, smart chip, or any other device or arrangement of devices that is capable of implementing the steps of the processes of the invention.

In various embodiments, the control system may also include or control a DC voltage module configured to bias the cathode and/or the anode to thereby create the electrolysis zone for hair styling. As mentioned above, the polarity on one or both electrodes may be controlled by the user via, e.g., a control on the handpiece itself that interfaces with the control system. The DC voltage module may be coupled directly to the anode and/or the cathode (e.g., via electrical wiring and/or components). The DC voltage module may also include a power supply, such as a battery, or other means to connect to an external power supply (such as a wall outlet). In various embodiments, the DC voltage module may include one or more rechargeable batteries, for example, Li-ion coin cells, 9-volt cells, D cells, or any other suitable cells, disposed in series within a sealed container, for example, a cylindrical container to form the power supply.

In additional embodiments, the styling system may supply a liquid styling agent that is already rich in hydroxide ions in addition to or instead of performing electrolysis in the handpiece. For example, the styling agent may include, consist essentially of, or consist of an alkaline liquid such as sodium hydroxide. Application of the styling agent to a bundle of hair with the handpiece enables styling of the hair. In such embodiments, the handpiece may lack one or both electrodes and may simply include one or more pads for application of the styling agent to hair within the handpiece. In various embodiments, the flow of the fluid to and through the handpiece may be controlled (by, e.g., the control system) in response to one or more sensors in or proximate the handpiece that detect ionic resistance or conductivity to ensure sufficient flow of the styling agent for the desired hair styling.

In various embodiments, the handpiece may be utilized to straighten hair via application of an alkaline styling agent, followed by neutralization of the alkaline styling agent by application of a pH-neutralizing solution. In an exemplary technique, a bundle of hair is clamped between the two sides of the handpiece and drawn through the handpiece while the alkaline styling agent is applied to the hair within the handpiece. As detailed above, the styling agent may be alkaline as a result of electrolysis occurring within the handpiece itself (e.g., within the “electrolysis box”) or remotely in a separate holding tank fluidly connected to the handpiece. In other embodiments, an alkaline solution may simply be pumped into the handpiece from an external reservoir. In various embodiments, the bundle of hair is straightened via multiple passes (or “strokes”) of the handpiece. For example, in various embodiments at least two (e.g., 4-5 or more, or even 10-12 or more) strokes of the handpiece are applied to the bundle of hair to straighten it. In various embodiments, an indicator on the system (e.g., on the handpiece) may indicate the number of strokes applied to the bundle of hair. Each stroke may be counted when the handpiece is opened, after a stroke, repositioned, and then closed again on the bundle of hair.

After the hair is straightened, alkaline solution remnant on the bundle of hair may be neutralized via application of a pH-neutralizing (e.g., acidic) solution via the handpiece. For example, the bundle of hair may be clamped between the two sides of the handpiece and drawn through the handpiece while the neutralizing solution is applied to the hair within the handpiece. In various embodiments, the number of neutralizing strokes applied to the bundle of hair may be about the same number of strokes (e.g., more than two, 4-5 or more, or even 10-12 or more) utilized for the initial straightening of the bundle of hair. As detailed above, in various embodiments the neutralizing solution may be the same electrolyte as that used to straighten the hair, but the electrode polarity utilized to electrolyze the solution may be reversed to form a solution with an opposite pH. In other embodiments, a different acidic solution may be applied to the hair for neutralization.

Embodiments of the invention may include one or more aspects detailed in, and/or be utilized to style and/or straighten hair in accordance with techniques described in the '056 patent and the '1312 application.

The leak performance of a device in accordance with embodiments of the invention, specifically featuring vacuum-induced removal as detailed herein, was compared to a control device without such leakage-prevention components. The device in accordance with embodiments of the invention, operating at an average flow rate of styling agent of approximately 160 ml/min, exhibited a leak rate of the styling agent out of the handpiece of 0.3 grams/min or less. The leakage rate of the control device was approximately 33 grams/min; thus, the device in accordance with embodiments of the invention exhibited two orders of improvement in leakage rate. These results demonstrate the superior performance of devices in accordance with embodiments of the present invention.

### Hair Coloring

Embodiments of the present invention may also be utilized to permanently or semi-permanently color hair, even if hair straightening or other styling is not desired. For typical permanent hair coloring, two formulations are utilized. The first formulation is a color formulation, which may include various components including a pre-pigment, a coupler molecule, an ammonium compound, and others (e.g., conditioners, color preserving chemicals, thickener, fragrance, etc.) The second formulation is a developer formulation, which may include various components including an oxidizing agent (e.g., H₂O₂), an alkaline agent (e.g., NaOH), and other optional components. In a coloring process utilizing the two formulations, the two formulations are freshly mixed and applied to the hair, which is then washed after a waiting period (e.g., 30 minutes). During permanent hair coloring, the ammonium compound releases ammonia at the alkaline pH, which together with the alkaline pH opens the hair structure for the pre-pigment, coupler, and oxidizer. Inside, the hair oxidizer reacts with the pre-pigment and converts it into the pigment. The coupler binds with the pigment to form a larger molecule. Also, various components bind to certain amino acids in the hair protein to form crosslinks. For example, the dye precursor p-phenylenediamine (PPD) oxidizes to p-phenylenediamine and becomes reactive with other aromatic amino acids and the coupling agent. The larger molecules formed via these reactions within the hair are resistant to being washed out and hence produce shampoo-stable coloring in the hair.

For typical semi-permanent hair coloring, a single formulation is typically utilized. The formulation may include, for example, an acidic dye or combination of dyes blended to produce a desired color, a polyquaternium compound, a pore-filling molecule such as a peptide, and other optional components. In a semi-permanent hair-coloring process, the formulation applied to the hair, incubated for a waiting period (e.g., about 30 minutes), and then the hair is washed. Such semi-permanent hair-coloring treatments typically resist a number of shampoo washes (e.g., four to eight washes). During semi-permanent hair coloring, ionic complexes are formed. For example, a quaternary ammonium compound has a nitrogen atom carrying positive charge independent of the pH of the medium. A polyquaternium compound has multiple such N⁺ groups. The polyquaternium compound binds the hair that is negatively charged at neutral pH. The acidic dyes with negatively charged groups bind on the polyquaternium compound and thus color the hair.

Devices and handpieces in accordance with embodiments of the invention may be utilized to color hair, permanently or semi-permanently, even if hair straightening or other styling is not desired. That is, devices in accordance with embodiments of the invention may be modified such that, even during application of voltage to form an electrolysis zone, application of the handpiece to the hair results in coloring rather than straightening or other electrolysis-induced styling. As detailed herein, in various embodiments when the handpiece is applied to hair, the electric field or current induces motion mainly of small ions such as Na⁺, Li⁺, H⁺, CO₃²⁻, OH⁻, etc., rather than charged macromolecules. That is, the small ions carry the most of the current, and charged macromolecules may only drift slowly in the electrolyte; hence, typically devices in accordance with embodiments of the invention does not induce motion of dye molecules or other organic molecules into the hair. However, when the device is applied to hair, it dynamically creates a high pH on the hair, and this causes a significant swelling of the hair strands. When the hair is swelled and immersed in or wet by an electrolyte, it invariably imbibes or absorbs a large amount of the electrolyte formulation. Thus, in various embodiments of the invention, dye molecules are saturated into the electrolyte and significantly penetrate the hair during application of the device.

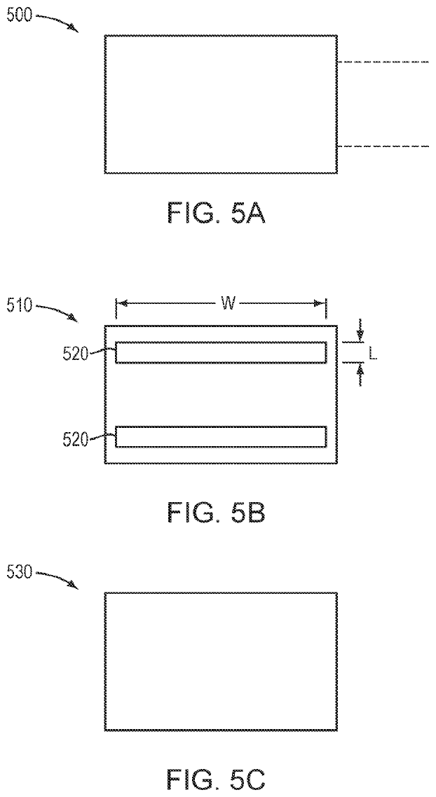

In various embodiments, a two-layer mask is applied to one of the electrodes of the device, e.g., the cathode, in order to enable hair coloring while avoiding undesired straightening or styling. FIG. 5A is a plan view schematic of the cathode 500 of the device, as detailed above. FIGS. 5B and 5C are plan view schematics of the layers of the two-layer mask that may be applied to the cathode 500. Mask layer 510 is sized and shaped to cover the cathode 500 and defines one or more windows 520 therethrough. In various embodiments, the mask layer 510 includes, consists essentially of, or consists of an electrical insulator (e.g., a plastic, ceramic, etc.) and thus largely does not allow flow of electrical current thereacross. During operation of the device, the electrical current is allowed to flow through the window(s) 520, which therefore concentrate the current in one or more locations. While FIG. 5B depicts the mask layer 510 as defining two windows 520, in various embodiments, the mask layer 510 defines only a single window 520, and that window may be oriented to be closer to the scalp (and thus the roots of the hair) of the subject during use of the handpiece. In various embodiments, the width W of the window 520 extends substantially (e.g., at least 70%, at least 80%, or at least 90%) across the width of the cathode 500. In various embodiments, the length L of the window 520 ranges from, for example, approximately 2 mm to approximately 4 mm. In various embodiments, the one or more windows 520 may expose only a fraction of the surface of the cathode 500, e.g., less than approximately 10% or less than approximately 20%.

Mask layer 530 is sized and shaped to fit over mask layer 510 and in various embodiments does not define any windows or openings therethrough. In various embodiments, the mask layer 530 includes, consists essentially of, or consists of an absorbent material such as cloth, sponge, membrane, etc., and therefore allows the electrolyte from the handpiece to be absorbed and soak into the mask layer 530. The mask layer 530 serves to space the hair away from the cathode 500, and in various embodiments may have a thickness ranging from approximately 50 microns to approximately 350 microns, or even more.

In various embodiments, the combination of mask layers 510, 530 enables pigments to enter the hair while resisting straightening or other undesired hair styling during use of the device. As mentioned above, where the electrical current is active, pigments are generally unlikely to enter the hair. As smaller ions, which have higher charge density, are preferentially located near the cathode, during use of the device the electrical current creates a gradient of the hair dye, which tends to repel the dye away from the electrodes. However, the pH-induced hair swelling occurs preferentially close to the cathode 500. Thus, use of the mask layer 510 limits the electrical current and focuses it through the window 520 proximate the edge of the cathode 500. When exposed to the current through the window 520, the hair begins to swell, and then, as the handpiece is swept along the hair, the dye and electrolyte mixture absorbed into the mask layer 530 is allowed to be absorbed into the hair due to the blocking of the electrical current by the remainder of the mask layer 510.

Moreover, the mask layer 530 also acts as a spacer layer to maintain a distance between the hair and the cathode 500 to a sufficient amount to prevent straightening or styling during the application of color to the hair. In various embodiments, the pH of the styling zone formed over the cathode 500 is highest (e.g., 13-14) near the surface of the cathode 500 and decreases with increasing distance away from the cathode surface. Thus, the thickness of the mask layer 530 is sufficient to maintain the hair at a sufficient distance from the cathode 500 such that it is exposed to a pH less than the pH of 13-14 at which straightening of the hair occurs. The hair is still exposed to a basic pH sufficient to swell the hair, and therefore enable absorption of the hair color, but insufficient to straighten or style the hair.

Various different electrolytes may be utilized during hair coloring processes in accordance with embodiments of the invention. A bleaching electrolyte may be utilized for bleaching or lifting the existing natural or artificial color of the hair. In various embodiments, a bleaching electrolyte may include, consist essentially of, or consist of water, guanidine, an organic acid (e.g., citric acid and/or maleic acid), hydrogen peroxide, and propylene glycol. Guanidine is an organic base that conjugates with the organic acid to create the electrolyte. Guanidine is a strong chaotropic agent that causes denaturation of the protein and opening of the hair lattice for the other agents to diffuse deeper inside. The organic acid such as maleic acid and/or citric acid reduces damage to the hair during the processing. Guanidine dissolves in water and/or the electrolyte to give a basic solution and addition of the organic acid brings the electrolyte closer to a neutral pH. Hydrogen peroxide is a bleaching agent, and the propylene glycol is a humectant.

A coloring electrolyte may be utilized to color hair. In various embodiments, the coloring electrolyte may include, consist essentially of, or consist of water, guanidine, an organic acid (e.g., citric acid and/or maleic acid), sodium and/or potassium laurate, a pigment or pigment blend, a polyquaternium compound, trimethyl glycine, propylene glycol, a UV absorbent and/or sunscreen, and an antioxidant. In various embodiments, one or more components may be omitted.

Guanidine is an organic base that conjugates with the organic acid to create the electrolyte. Guanidine is a strong chaotropic agent that causes denaturation of the protein and opening of the hair lattice for the pigments/agents to diffuse deeper inside. Also, guanidine left behind in the hair after the process may complex with acidic dye to keep it stable after coloring against hair washes. In various embodiments, guanidine-based electrolyte is less damaging to hair compared to sodium/potassium/lithium-based inorganic salts, as in coloring embodiments of the invention, the intention is to raise the pH only to a level to enable hair coloring but insufficiently high to cause straightening. In various embodiments, an organic electrolyte prevents the precipitation of the pigments to be dissolved in the electrolyte. As mentioned above for the bleaching electrolyte, the organic acid such as maleic acid and/or citric acid reduces the damage to the hair during the processing. Guanidine dissolves in water to give a basic solution and addition of the organic acid brings the electrolyte closer to the neutral pH. The sodium and/or potassium laurate is the agent that locks out the pigments during neutralization. During neutralization, the hair is treated with an acidic conditioner (e.g., a conditioner with high concentration of citric acid). Neutralization of the hair may also be achieved by changing the polarization of the treating electrode in the device from cathode to anode and this precipitates the lauric acid (a hydrophobic oil molecule) as semi-solid waxy substance, increasing the hydrophobicity of the hair and hence decreasing the penetration of water deep into the hair during subsequent hair shampoo washes after coloring to increase the life of the color. The polyquaternium compound is a quaternary ammonium polymer compound that conditions the hair and complexes and holds the acidic dyes. Trimethyl glycine is a quaternary ammonium compound that penetrates deeper into the hair and complexes with the acidic dyes to keep the color stable against hair washes. Propylene glycol is a humectant that prevents the electrolyte from drying out on the hair during the coloring process. The UV absorbent or sunscreen protects against light-induced fading of the color, while the antioxidant protects against oxidative de-colorization.

Various embodiments of the invention utilize permanent pigments based on dye precursors such as p-phenylenediamine (PPD) and p-toluenediamine (PTD). The dye may be utilized with couplers like resorcinol and naphthols. In various embodiments, after washing and towel drying, the hair is first combed with the device infused with the hydrogen peroxide-based bleaching electrolyte, followed by the dye electrolyte (which may include the pre-pigments p-phenylenediamine (PPD) and p-toluenediamine (PTD) and the couplers as mentioned above). In various embodiments, no washing is performed between the bleaching electrolyte treatment and the coloring electrolyte treatment. During treatment, the pre-dye/pre-pigment molecule is oxidized inside the hair and crosslinks with the coupler molecule and aromatic amino acids in the hair protein to become stabilized inside the hair. After application of the coloring electrolyte, the hair may be allowed to incubate for a waiting period (e.g., approximately 30 minutes). In various embodiments, a neutralizer may then be applied to the hair with the device, as detailed herein, and then the hair may be washed to remove any excess colorants.

In various embodiments, for semi-permanent hair coloring, after washing and towel drying, the hair may first be treated with the bleaching electrolyte, washed thoroughly, and then treated with the coloring electrolyte. After application of the coloring electrolyte, the hair may be allowed to incubate for a waiting period (e.g., approximately 30 minutes). In various embodiments, a neutralizer may then be applied to the hair with the device, as detailed herein, and then the hair may be washed to remove any excess colorants.

Various embodiments of the invention may utilize one or more pigments utilized for food coloring, which are highly water-soluble and safe. Such pigments include, but are not limited to, E104: Quinoline Yellow, E122: Carmoisine, E124: Ponceau 4R, E131: Patent Blue V, E142: Green S, FD&C Blue No. 1—Brilliant Blue FCF, E133 (blue shade), FD&C Blue No. 2—Indigotin, E132 (indigo shade), FD&C Green No. 3—Fast Green FCF, E143 (turquoise shade), FD&C Red No. 3—Erythrosine, E127 (pink shade), FD&C Red No. 40—Allura Red AC, E129 (red shade), FD&C Yellow No. 5—Tartrazine, E102 (yellow shade), and FD&C Yellow No. 6—Sunset Yellow FCF, E110 (orange shade). Other semi-permanent hair dyes may also be utilized, such as basic blue 99, basic brown 16, acid violet 43, basic red 76, basic yellow 57, basic brown 17, basic red 51, and green 25 (Alizarin Cyanine Green F). Blending of two or more pigments may be performed to form compound colors or shades.

In various embodiments, different base/primary coloring pigments added to achieve a desired compound color mix may have different masses, diameters, and/or charges. Depending on these properties, the base/primary color pigments may diffuse at different rates to the hair. Thus, various pigment blends may have higher concentrations of those slow-diffusing molecules to achieve final desired compound color, and the actual physical pigment blend may therefore not have the exact desired color for the hair (i.e., the color that will result upon treatment of the hair with the blend). For example, if the desired color is a blend of blue and red, and the red pigment has larger, slow-diffusing molecules, the blend may incorporate a higher concentration of the red pigment to optimize the actual hair color resulting from treatment with the blend.

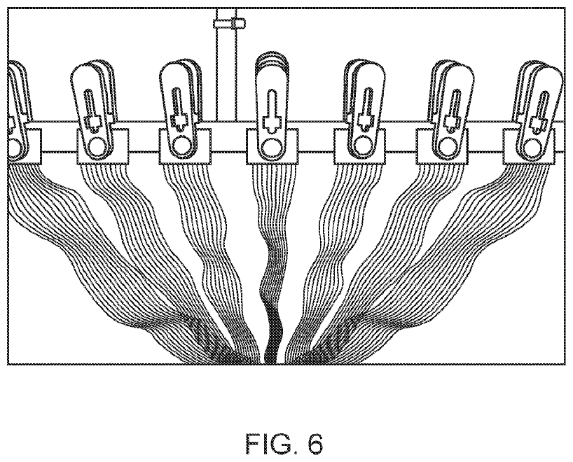

FIG. 6 depicts nine different hair strands successfully colored with different colored dyes in accordance with embodiments of the invention. As shown, the hair strands retained their natural shape and were not straightened or otherwise styled during application of the hair color.

The terms and expressions employed herein are used as terms of description and not of limitation, and there is no intention, in the use of such terms and expressions, of excluding any equivalents of the features shown and described or portions thereof, but it is recognized that various modifications are possible within the scope of the invention claimed.

1. A hairstyling device comprising:
an anode-side housing having an outer perimeter;
an electrically conductive anode disposed within the anode-side housing;
a cathode-side housing having an outer perimeter;
an electrically conductive cathode disposed within the cathode-side housing;
a membrane disposed between the anode and the cathode;
an absorbent pad disposed adjacent to the membrane;
one or more fluid inlets for supplying a liquid styling agent from one or more reservoirs to the absorbent pad;
one or more fluid outlets for drawing the liquid styling agent back into the one or more reservoirs;
a vacuum source coupled to the one or more fluid outlets, the vacuum source being configured to remove liquid styling agent that is unabsorbed by the pad; and
a power module for supplying electrical power to the anode and/or the cathode to thereby form an electrical potential difference between the anode and the cathode,
wherein, in an open configuration, the anode-side housing and the cathode-side housing are spaced apart, and
wherein, in a closed configuration, the anode-side housing and the cathode-side housing are in direct contact to enclose therewithin an electrolysis zone, whereby hair within the electrolysis zone contacts the styling agent and is styled via electrolysis resulting at least in part from the potential difference.

2. (canceled)

3. The device of claim 1, further comprising:
an anode-side foam seal disposed along the outer perimeter of the anode-side housing; and
a cathode-side foam seal disposed along the outer perimeter of the cathode-side housing.

4. The device of claim 3, further comprising:
an anode-side vacuum line fluidly connecting the anode-side foam seal to a second vacuum source; and
a cathode-side vacuum line fluidly connecting the cathode-side foam seal to the second vacuum source.

5.-6. (canceled)

7. The device of claim 3, further comprising:
a first solid seal disposed along an outer edge of the anode-side foam seal to prevent lateral flow of styling agent from the anode-side foam seal; and
a second solid seal disposed along an outer edge of the cathode-side foam seal to prevent lateral flow of styling agent from the cathode-side foam seal.

8. The device of claim 1, further comprising a foam seal disposed along the outer perimeter of the anode-side housing or along the outer perimeter of the cathode-side housing.

9. The device of claim 8, further comprising a vacuum line fluidly connecting the foam seal to a second vacuum source.

10.-11. (canceled)

12. The device of claim 1, further comprising a control system for controlling the power module and flow of the styling agent.

13. The device of claim 12, further comprising one or more sensors for detecting when the hairstyling device is in the open configuration and/or in the closed configuration.

14. (canceled)

15. The device of claim 13, wherein the control system is configured to supply the styling agent only when the hairstyling device is in the closed configuration.

16. The device of claim 12, wherein the control system is configured to regulate flow of the styling agent based at least in part on (i) an ionic conductivity between the anode and the cathode and/or (ii) a temperature of the styling agent.

17. The device of claim 1, wherein the control system is configured to reverse a polarity of the potential difference formed by the power module.

18.-82. (canceled)

83. A method of coloring hair, the method comprising:
providing a device comprising (i) a first housing containing a first electrode, (ii) a second housing containing a second electrode, (iii) disposed over the second electrode, a first masking layer comprising an electrically insulating material and defining a window therethrough, and (iv) disposed over the first masking layer, a second masking layer comprising an absorbent material, wherein (a) in an open configuration, the first housing and the second housing are spaced apart, and (b) in a closed configuration, the first housing and the second housing are in direct contact to enclose therewithin a treatment zone;
enclosing a hair bundle within the treatment zone;
supplying a bleaching electrolyte within the treatment zone;
pre-treating the hair bundle by contacting the hair bundle with the bleaching electrolyte, and moving the device along a length of the hair bundle;
thereafter, supplying a coloring electrolyte within the treatment zone; and
coloring the hair bundle by contacting the hair bundle with the coloring electrolyte, and moving the device along the length of the hair bundle.

84. The method of claim 83, further comprising, after coloring the hair bundle, neutralizing coloring electrolyte remaining on the hair bundle by (i) supplying an acidic solution within the treatment zone, and (ii) contacting the hair bundle with the acidic solution, and moving the device along the length of the hair bundle.

85. The method of claim 83, further comprising washing the hair bundle, before coloring the hair bundle, to remove bleaching electrolyte therefrom.

86. The method of claim 83, further comprising washing the hair bundle, after coloring the hair bundle, to remove coloring electrolyte therefrom.

87. The method of claim 83, wherein, during at least one of pre-treating or coloring the hair bundle, (i) an electrolyte pH is at least 13 away from the second masking layer and (ii) the electrolyte pH is less than 13 on the second masking layer.

88. The method of claim 83, further comprising preventing leakage of the bleaching electrolyte with a first foam seal disposed along an outer perimeter of the first housing, a second foam seal disposed along an outer perimeter of the second housing, or both.

89. The method of claim 88, wherein at least one of the first foam seal or the second foam seal comprises, at least in part, an open-cell foam.

90. The method of claim 89, wherein preventing leakage of the bleaching electrolyte comprises removing bleaching electrolyte from the first foam seal and/or the second foam seal with a vacuum source.

91. The method of claim 83, further comprising preventing leakage of the bleaching electrolyte by removing excess bleaching electrolyte from the first housing and/or the second housing with a vacuum source.

92.-100. (canceled)
